(19) United States
(12) Patent Application Publication
(10) Pub. No.: US 2002/0001590 A1
(43) Pub. Date: Jan. 3, 2002

(54) Antibacterial therapy for multi-drug resistant bacteria

(75) Inventors: Max Kelly, Toronto (CA); Allison McGeer, Toronto (CA); Barbara M. Willey, Toronto (CA)

Correspondence Address: MERCHANT & GOULD PC, P.O. BOX 2903, MINNEAPOLIS, MN, 55402-0903, US

(73) Assignee: Mount Sinai Hospital

(21) Appl. No.: 09/834,162

(22) Filed: Apr. 12, 2001

Related U.S. Application Data
(60) Provisional application No. 60/198,723, filed on Apr. 20, 2000.

(51) Int. Cl. A01N 63/00; A61K 39/00; A61K 39/38; C12N 15/00; C12N 15/09; C12N 15/63; C12N 15/70; C12N 15/74

(52) U.S. Cl. 424/184.1; 435/320.1

(57) ABSTRACT

The invention relates to selected bacteriophages, formulations containing same, and their use in killing or inhibiting the growth of bacteria, particularly methicillin-resistant Staphylococcus aureus (MRSA).

### FIELD OF THE INVENTION

The invention relates to selected bacteriophages, formulations containing same, and their use in killing or inhibiting the growth of bacteria.

### BACKGROUND OF THE INVENTION

The rise in the incidence of multi-drug resistant bacterial infections has made the need for alternative means of treatment more pressing. In particular, the number of nosocomial infections due to antibiotic resistant bacteria has increased sharply in recent years. Methicillin-resistant Staphylococcus aureus (MRSA) has emerged as one of the main causes of such infections [Voss, A, and B. N. Doebbeling, International Journal of Antimicrobial Agents 5, 1995, 101-106; McGeer, A., et al,. LPTP Newsletter, 1996 (190): p. 1-4]. NRSA infections are normally combated with the administration of the glycopeptide antibiotic, vancomycin. There have been reports of the development of vancomycin intermediate Staphylococcus aureus (VISA) infections in patients being treated with vancomycin for NRSA infections. This strongly suggests that the continued use of vancomycin to treat MRSA infections could give rise to a fully glycopeptide resistant population of Staphylococcus auerus [Smith, T. L., New. Eng. J. Med. 1999, 340 7): p. 493-501]. Furthermore, while other antibiotics were used to eradicate the infections, the resulting complications proved fatal in all the reported cases. Therefore, there is a need for an alternative approach to the treatment of antibiotic resistant infections.

Therapy using bacteriophages is based on the principle of administering phages capable of killing the bacteria which are the cause of the infection [Parker, M. T. Methods in Microbiology Vol. 7B, 1972, New York and London: Academic Press]. The phages infect, replicate and then lyse the target bacteria without affecting the patient's tissues or the normally occurring micro-flora. Phage therapy offers the prospect of an adaptive model of treatment well suited to fighting antibiotic resistant infections [Smith, H. W., a. H., M. B. J. of Gen. Microbiol., 1982, 128 ((Pt 2)): p.307-318; Merril, C. R., et al, Proc. Natl. Acad. Sci. USA, 1996, 93(8): p. 3188-3192).

The effectiveness of phage therapy was demonstrated against E. coli and S. typhimurium in animal models of infection (mice, calves and piglets) [Smith, supra; Meril, supra). A number of studies of phage therapy in animal models of infection have been carried out targeting E. coli, Salmonella typhimurium, Pseudomonas, and Staphylococcus with a variety of results, mostly positive. However, these studies did not target clinically relevant strains, particularly those strains implicated in human diseases.

### SUMMARY OF THE INVENTION

The present inventors have studied the potential of phage therapy for treating antibiotic resistant bacteria. In particular, the present inventors have identified specific bacteriophages which virulently lyse MRSA. The inventors have significantly demonstrated that phages can be used to lyse a broad range of clinically relevant strains of multi-drug or antibiotic resistant bacteria.

Therefore, the present invention relates to bacteriophages selected from the species Myoviridae for use as active therapeutic substances, particularly in the treatment of infectious diseases caused by bacteria, preferably antibiotic resistant bacteria.

The invention also relates to formulations comprising isolated and purified bacteriophages from the species Myoviridae.

Further, the invention provides a method for treating an infectious disease caused by bacteria, preferably antibiotic resistant bacteria, in an animal comprising administering to an animal in need of such treatment a bacteriophage selected from the species Myoviridae. The invention also provides a method of reducing virulence of bacteria, preferably antibiotic resistant bacteria in a subject comprising administering to the subject an effective amount of a bacteriophage selected from the species Myoviridae.

Methods for killing or inhibiting the growth of bacteria are also provided comprising contacting the bacteria with an effective amount of a formulation of the invention. A medium that can be treated by this method may be a food product, substances used in making food products, medical instruments, skin, surgical implants, or metallic, plastic, tile, porcelain, or glass surfaces. The medium may be an inert carrier and such a formulation may be used in a conventional bactericide manner.

The invention also provides a novel bactericide prepared with one or more isolated and purified bacteriophages from the species Myoviridae for disinfecting or sterilizing anything to be protected against infection with pathogenic bacteria, including but not limited to food products, substances used in making food products, areas where there is preparation of foodstuffs, surgical implants, metallic, plastic, tile, porcelain, or glass surfaces, medical devices and instruments, and skin.

Strains of the sub species Twort that are capable of lysing about 99% of MRSA strains, (for example, C-MSRA1 to C-MSRA4 inclusive, Belgian, Swiss, and EMRSA1 to EMRSA inclusive), are particularly useful in the formulations and treatments of the invention. In preferred embodiments of the invention the formulations and methods include one or more of the following phages: φ812, φ131, SK311, Mu50, and U16. These bacteriophage have extremely high specificity for MRSA.

The formulations, compositions, bactericides, and methods of the invention are useful against strains of pathogenic bacteria, particularly multi-drug resistant bacteria. For example, they are suitable against strains of staphylococci such as Staphylococcus aureus and Staphylococcus epidermidis. The formulations and methods are particularly useful against strains of staphylococci that are of reduced sensitivity to glycopeptides such as vancomycin or teicoplanin. In an embodiment of the invention, the staphlopcocci strains are also methicillin resistant.

These and other aspects, features, and advantages of the present invention should be apparent to those skilled in the art from the following drawings and detailed description.

### DETAILED DESCRIPTION OF THE INVENTION

In accordance with the present invention there may be employed conventional molecular biology and microbiology techniques within the skill of the art. Such techniques are explained fully in the literature. See for example, Sambrook, Fritsch, & Maniatis, Molecular Cloning: A Laboratory Manual, Second Edition (1989) Cold Spring Harbor Laboratory Press, Cold Spring Harbor, N.Y; and Parker, M. T. Methods in Microbiology Vol. 7B, 1972, New York and London: Academic Press.

Bacteriophages that can be used in the methods of the invention are strains that are capable of doing direct or indirect harm to the bacteria. Suitable bacteriophages may include lytic bacteriophages, bacteriophages that are lysogenic and later become lytic (e.g. phages genetically modified to become lytic), and nonlytic bacteriophages that produce products that are harmful to the bacteria. Preferably lytic bactiophages are used in the present invention.

Bacteriophages that can be used in the formulations and methods of the invention include those belonging to the family Myoviridae [Pantucek, R. et al, Virology 1998, 245(2): p. 241-252). In particular, strains of the species Twort that are capable of lysing about 99% of MRSA strains may be used in the present invention. In a preferred embodiment of the invention the formulations and methods include one or more of the following bacteriophages: φ812, φ131, SK311, and U16.

A bacteriophage may be modified to enable the bacteriophage to delay inactivation by any and all parts of the host defense system that may reduce the numbers of bacteriophage and/or the efficiency of the bacteriophage at killing the host bacteria in an infection. Modified bacteriophages that are able to delay inactivation by the host defense system can be obtained by selection of modified strains by serial passage of the phage, or by genetic engineering of a phage, so that the modified phage will remain active in the body for longer periods of time than the wild-type phage. (See U.S. Pat. Nos. 5,811,093 5,766,892, 5,688,501.)

The bacteriophages can be used in combination with other anti-bacterial agents. Suitable antibacterial agents that can be used in combination with the bacteriophages include but are not limited to antibiotics and chemotherapeutic agents. Examples of such agents include the penicillins, cephalosporins, glycopeptides (e.g. vancomycin), aminoglycosides (e.g. amikacin, tobramycin), imipenem, erythromycin, carbapenems (WO9920638), penicillinase-resistant penicillin, anthraquinone derivatives, clavulanic acid, or combinations thereof (WO9622105).

The bacteriophage can be grown using appropriate bacteria (e.g. SA812) in suitable media. The resulting lysates are treated to provide a preparation that has no live organisms and toxins such as bacterial cell wall. For example, the resulting lysates can be sterilized using conventional methods such as filtration, and purified, using for example ultrafilitration, to remove bacterial cell wall. The bacteriophages, or formulations thereof as described herein, can be packaged and sealed into ampoules or otherwise prepared and packaged for administration. Approximate titers can be determined by checking the dilution that would produce lysis after coinnoculation with specific numbers of bacteria of standard test strains, and each batch can be tested for any surviving bacterial contaminants. In an embodiment, preparations with a minimum concentration of between 10⁸ and 10⁹ pfu are prepared. Where the bacteriophage is to be injected, it can be concentrated (e.g. lyophilized) and resuspended in buffers such as physiological saline. The bacteriophage preparations can be tested for toxicity, (e.g.in laboratory animals such as guinea pigs) to ensure that no residual bacterial surface fragments are present.

Accordingly, the bacteriophages may be formulated into compositions or formulations for administration to subjects in a biologically compatible form suitable for administration in vivo. By “biologically compatible form suitable for administration in vivo” is meant a form of the active substance to be administered in which any toxic effects are outweighed by the therapeutic effects. The active substances may be administered to animals including humans, domestic pets, livestock, pisciculture, zoo animals, and animals in aquatic parks. Administration of a therapeutically active amount of a formulation of the present invention is defined as an amount effective, at dosages and for periods of time necessary to achieve the desired result. For example, a therapeutically active amount of a substance may vary according to factors such as the disease state, age, sex, and weight of the individual. Dosage regima may be adjusted to provide the optimum therapeutic response. For example, several divided doses may be administered daily or the dose may be proportionally reduced as indicated by the exigencies of the therapeutic situation.

The dosage may be in the range of about 10⁶ to about 10³ pfu/kg/day, preferably about 10⁸ to 10¹¹ pfu/kg/day. The bacteriophage can be administered until successful elimination of the pathogenic bacteria is achieved.

The active substance may be administered in a convenient manner such as by injection (subcutaneous, intravenous, etc.), oral administration, pulmonary (e.g. aerosol or by other devices for delivery to the lungs), nasal spray, intramuscular, intraperitoneal intrathecal, intravitreal, vaginal, rectal, topical, lumbar puncture, and direct application. Depending on the route of administration, the active substance may be coated in a material to protect the substance from the action of enzymes, acids and other natural conditions that may inactivate the substance (e.g. enteric coated tablets or pills). The bacteriophage may be incorporated into an aerosol formulation specifically adapted for aerosol administration to the lungs by inhalation. Suitable means for aerosol administration are well known in the art and include the Proventil™ inhaler (Schering-Plough). The types and concentrations of the propellants in the device are adjusted based on the type of phage.

The pharmaceutical formulations described herein can be prepared by per se known methods for the preparation of pharmaceutically acceptable formulations which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the formulations include, albeit not exclusively, solutions of the active substances in association with one or more pharmaceutically acceptable vehicles or diluents, and contained in buffered solutions with a suitable pH and iso-osmotic with the physiological fluids.

Excipients which can be used as delivery vehicles are apparent to those skilled in the art.

The bacteriophage can be lyophilized and dissolved prior to administration by intravenous injection. The bacteriophage can be dissolved in a suitable carrier for example an aqueous solvent or buffer or suspended in any suitable liquid, colloidal, or polymeric matrix to create bactericides. The bactericides can be incorporated into ointments, or coatings for medicinal uses such as the treatment of infections as described herein, wound dressings, or surgical implants, and as a broad spectrum disinfectant for skin or oral rinses, disinfectant scrubs, wipes, or lotions. The bactericides can be used for cleaning medical instruments, in pre-operative surgical scrubs, and the like.

The formulations and methods of the invention are suitable against strains of staphylococci for example, Staphylococcus aureus and Staphylococcus epidermidis. The formulations and methods are particularly useful in the prevention and treatment of infections caused by strains of staphylococci which are of reduced sensitivity to glycopeptides such as vancomycin or teicoplanin. In an embodiment of the invention, the staphlopcocci strains are also methicillin resistant. In a preferred embodiment of the invention the compositions and methods are used to prevent and treat MSRA infections, in particular those caused by C-MSRA1 to C-MSRA4 strains inclusive, Belgian strain, Swiss strain, and EMRSA1 to EMRSA strains inclusive.

The foregoing embodiments of the invention are further described in the following example. However, the present invention is not limited by the Example, and variations will be apparent to those skilled in the art without departing from the scope of the present invention.

The following non-limiting example is illustrative of the present invention:

### EXAMPLE

Phage therapy for the treatment of methicillin-resistant Staphylococcus aureus (MRSA) was developed by first identifying bacteriophages capable of lysing a selection of MRSA isolates representative of the variations found in vivo. 8 candidate phages were identified which had been shown to be capable of killing a high percentage of Staphylococcus aureus strains:

phages 44AHJD, 2638A, Twort, P68, φ812, φ131, SK311, and U16. The selected isolates were representative of the variations in phage typing patterns and each of the four clones responsible for the majority of MRSA infections in Ontario.

Methods and Materials:

Selection and Propagation of Bacteriophages:

Samples of the phages Twort, 2638A, P68, and 44AHJD and their bacterial propagating strains were obtained from Dr. H. W. Ackermann, director of the Felix d'Herelle Reference Centre for Bacterial Viruses at the University of Laval. Samples of the phages φ812, φ131, SK311, and U16 were obtained from Dr. L. Valicek of the Czech Collection of Animal Pathogens at the Veterinary Research Institute, Brno, Czech Republic. Upon receipt, each propagating strain was sub-cultured onto a Columbia base blood agar (5% sheep blood) plate (BA plate)(Oxoid) for purity and incubated overnight at 37° C. A single colony was used to inoculate 5 ml of trypticase soy broth (TSB)(Difco), which was incubated overnight at 37° C. with shaking. 1.5 ml of the overnight culture was added to 150 ml of TSB and grown at 37° C. with shaking for 3hrs. At the end of the 3hrs, by which point the cultures had become somewhat turbid with bacterial growth, 500 μl of the phage solutions (10⁵⁻⁸ pfu/ml) were added to the cultures. The cultures were left without shaking for 10-15 min to allow phage adsorption, and then grown under the same conditions until lysis was observed or 6hrs passed. The cultures were then divided into 50 ml Falcon tubes and centrifuged at 2000g for 20min to pellet the bacterial cells and debris. The supernatant was filtered through a 0.22 μm pore size vacuum driven filter (Millipore Stericup) to sterilise the solution and remove as much bacterial debris as possible. The final phage solution was titrated on lawns of the appropriate propagating strain, and the concentration calculated in plaque forming units per ml (pfu/ml) [Ackerman, H. W. a. D., M. S., Viruses of Prokaryotes Vol 1. 1987, Boca Raton, Florida: CRC Press].

Selection of MRSA Isolates:

The study targeted the four MRSA strains which have been responsible for most of the MRSA cases in Ontario for the last seven years (Ontario Epidemic, North American, British Empire and Historic). These strains have been delineated on the basis of macro-genetic analysis (Smal digestion and pulse field gel electrophoresis). A selection of 92 isolates, representative of each of the strains, were plated from freezer stocks maintained in the Microbiology Department of Mount Sinai Hospital, Toronto, Ontario. Their classification as methicillin-resistant was based on the determination of the minimum inhibitory concentration of a variety of antibiotics, in accordance with the guidelines set out by the National Centre for Clinical Laboratory Standards.

In addition, most of the isolates had been phage typed by the Laboratory Centres for Disease Control (Winnipeg, Manitoba, Canada) according to the standard protocols Parker, M.T., Methods in Microbiology, Vol 7B, 1972, New York and London: Academic Press). The isolates chosen represented a wide variety of phage types, including 20 classified as non-typable.

Finally, to test the specificity of phages, 5 coagulase-negative Staphylococcus aureus (CNST) clinical isolates, and ATCC strains of S. saprophyticus (ATCC #15305) and S. epidermidis (ATCC #12228) were also tested for their susceptibility.

Screening of MRSA Isolate Susceptibility to Phages:

The MRSA isolates were plated onto BA plates and incubated overnight at 37° C. Lawns of bacterial growth were created by making up 0.5 MacFarland standard solutions in sterile 0.9% NaCl solution (0.5 MacFarland is equivalent to 1.5×10⁸ cfu/ml). Sterile swabs soaked in the 0.5 MacFarland solutions were used to spread bacteria evenly across the plates. Dilutions of each phage were made up, ranging in concentration from 10⁹-10³, inclusively. 10 μl of each dilution was spotted onto the bacterial lawns and the solution allowed time to be absorbed. The plates were then incubated overnight at 37° C. The formation of plaques exhibiting confluent lysis was taken as evidence of virulent infection and successful lysis of the target MRSA strain (Parker, M. T., supra). The degree of lysis was classified as either not susceptible (no visible plaques), weakly susceptible (very few isolated plaques), or strongly susceptible (total lysis at higher concentrations, and clearly defined plaques at lower concentrations). The isolates were screened in batches of variable size (10-30) and control plates of the propagating strains were run in parallel with each batch to ensure the activity of the phage dilutions. One isolate which possessed a very strong capsule and which was initially resistant to the phages was re-tested on Trypticase Soy Agar (TSA).

Once all 92 isolates had been screened separately with each phage, a selection of MRSA isolates, representative of both the strongly susceptible and not susceptible groups, were tested with a combination of all four phages. 10 μl of a concentration of each phage were spotted together onto the same lawn, so that the concentration of each phage was the same as when tested individually.

Results:

92 MRSA isolates, representative of the four strains responsible for the majority of the MRSA cases in Ontario over the last 7 years, were screened with 4 phages from the family of Myoviridae. The results are shown in the table below.

Relative Susceptibility of MRSA Isolates to each Phage:

| | | Weakly | |
|---|---|---|---|
| | Resistant | Susceptible | Strongly Susceptible |
| Phage | (%) | (%) | (%) |
| 44AHJD | 29 (36.25) | 22 (27.5) | 29 (36.25) |
| P68 | 24 (30) | 24 (30) | 32 (40) |
| 2638A | 80 (100) | 0 (0) | 0 (0) |
| Twort | 80 (100) | 0 (0) | 0 (0) |
| φ812 | 6 (6.5) | 5 (5.5) | 81 (88) |
| φ131 | 2 (2.2) | 9 (9.7) | 81 (88) |
| SK111 | 5 (5.4) | 8 (8.7) | 79 (85.9) |
| U16 | 3 (3.2) | 11 (12) | 78 (84.8) |

The percentage of isolates of each strain that were susceptible was compared to determine whether the macro-genetic characteristics analysed by pulse field gel electrophoresis is a predictor of susceptibility to phages.

The isolate displaying the strong capsule formation that was initially resistant to phages φ812, φ131, SK311, and U16, was re-tested on TSA and found to be strongly susceptible.

Discussion:

Of the 8 phages, φ812, φ131, SK311, and U16 collectively proved capable of lysing ˜99% of the isolates screened, most of which were strongly susceptible. These 4 phages have therapeutic value against MRSA. Phage therapy's advantages over conventional antibiotics for the treatment of MRSA include its specificity of action, its relatively non-specific mechanism and its natural adaptability. While conventional antibiotics affect all bacteria, including the normal micro-flora, bacteriophages only destroy the host bacteria. Since there are no commensal strains of S. aureus, there would be no ill effects due to the elimination of such organisms as the non-pathogenic E. coli found in the gastrointestinal tract.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description. Such modifications are intended to fall within the scope of the appended claims.

All publications, patents and patent applications mentioned herein are incorporated herein by reference for the purpose of describing and disclosing the, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise.

We claim:

1. Isolated and purified bacteriophages from the species Myoviridae that are used in the treatment of infectious diseases caused by bacteria.

2. A pharmaceutical formulation comprising an isolated and purified bacteriophage from the species Myoviridae.

3. A pharmaceutical formulation as claimed in claim 2 wherein the bacteriophage is selected from the group consisting of φ812, φ131, SK311, and U16.

4. A method for treating an infectious disease caused by bacteria in an animal comprising administering to an animal in need of such treatment an isolated and purified bacteriophage as claimed in claim 1.

5. A method as claimed in claim 4 wherein the infectious disease is caused by methicillin-resistant Staphylococcus aureus (MRSA).

6. A method of reducing virulence of bacteria in a subject comprising administering to the subject an effective amount of an isolated and purified bacteriophage as claimed in claim 1.

7. A method as claimed in claim 6 wherein the bacteria is a methicillin-resistant Staphylococcus aureus (MRSA).

8. Methods for killing or inhibiting the growth of bacteria comprising contacting the bacteria with an effective amount of an isolated and purified bacteriophage as claimed in claim 1.

9. A method as claimed in claim 8 wherein the bacteria is a methicillin-resistant Staphylococcus aureus (MRSA).

10. A method as claimed in claim 8 wherein the method is used to treat a food product, a substance used in making a food product, a medical instrument, skin, a surgical implant, or metallic, plastic, tile, porcelain, or glass surface.

11. A bactericide comprising one or more isolated and purified bacteriophages from the species Myoviridae.

12. A bactericide as claimed in claim 11 comprising one or more of φ812, φ131, SK311, and U16.
